(19) United States
(12) Patent Application Publication
(10) Pub. No.: US 2006/0199980 A1
(43) Pub. Date: Sep. 7, 2006

(54) Process for preparing alkylene glycols

(75) Inventors: Achim Stankowiak, Altoetting (DE); Erwin Holzhauser, Ganghofen (DE); Alexander Snell, Altoetting (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/365,020

(22) Filed: Mar. 1, 2006

(30) Foreign Application Priority Data
Mar. 1, 2005 (DE) 102005009133.4

(51) Int. Cl. C07C 41/03

(52) U.S. Cl. 568/679

(57) ABSTRACT

The invention relates to a process for preparing alkylene glycols by hydration of alkylene oxides in the presence of polyalkylene glycol dialkyl ethers of the formula
R¹—O—[—(CH₂CH₂O)ₘ(CH(CH₃)CH₂)—O]ₙ—R² in which m=0-100, n=0-100, where n+m is at least equal to 1,
R¹ is a C₁- to C₆-alkyl radical,
R² is a C₁- to C₆-alkyl radical, where R² may be different from R¹, with the proviso that for at least 50 mol % of the polyalkylene glycol dialkyl ether m+n is greater than or equal to 11.

The present invention relates to a process for preparing alkylene glycols by hydrolyzing the corresponding alkylene oxides in the presence of a polyglycol dialkyl ether.

It is known from the prior art that alkylene oxides can be hydrolyzed to the corresponding alkylene glycols (Ullmann's Encyclopedia of Industrial Chemistry, Wiley-VCH Verlag, 6th edition, CD-ROM 2003). One disadvantage of the known processes is that a very large excess of water (the molecular ratio of alkylene oxide to water is from 1:6 to 1:20) is necessary in order to avoid as far as possible the formation of di-, tri- and polyalkylene glycols. The resulting aqueous crude alkylene glycol solution is concentrated in evaporators and fractionally distilled in a plurality of vacuum columns. This is associated with considerable expenditure on apparatus and energy and thus high costs. In addition, despite the large excess of water, the selectivity for example in the preparation of monoethylene glycol is only about 90%. Additional products are about 9% diglycol and 1% triglycol and higher ethylene glycols (see: K. Weissermel, H. J. Arpe “Industrielle Organische Chemie”, 5th edition, 1998, pages 167-168). There have been descriptions in the literature of a large number of processes which increase the desired selectivity or reduce the required amounts of water.

DE-A-29 24 680 describes a process for preparing alkylene glycols in which the catalytic hydrolysis is carried out in the presence of CO₂ via a glycol ester intermediate and in the presence of an organic solvent. Described solvents are esters, ketones and ethers, especially acetone and dioxane. Very high selectivities for monoethylene glycol of up to 99% are achieved in the described process, although with use of extremely large amounts of catalyst (0.22 mol of catalyst per liter of ethylene oxide), which lead to doubts about the efficiency of this process. A further disadvantage of this process is that compressed carbon dioxide must be fed in, which is associated with increased complexity of apparatus.

U.S. Pat. No. 4,760,200 describes a process in which the hydrolysis is carried out in the presence of an organic cosolvent, preferably 1,2-dimethoxyethane, and where appropriate of water-soluble metallate anions of group VI of the periodic table. The selectivities for monoethylene glycol are good, although the preferred solvent 1,2-dimethoxyethane is toxic and may have harmful effects both on fertility and on the unborn child.

A likewise metallate-catalyzed process is described in EP-A-01 56448. In this case, benzene, xylene, toluene, dichloromethane or 1,1,2-trichloroethane are employed as cosolvents in order to recycle the used catalyst.

The aim of the present invention is to eliminate the disadvantages mentioned. The invention was based on the object of developing a process for preparing alkylene glycols which makes it possible to carry out the process without an excess of water, or with only a small excess of water, while the selectivity for formation of monoalkylene glycols remains the same or is even increased.

The invention relates to a process for preparing alkylene glycols by hydration of alkylene oxides in the presence of polyalkylene glycol dialkyl ethers of the formula
R¹—O—[—(CH₂CH₂O)ₘ(CH(CH₃)CH₂)—O]ₙ—R²
in which
m=0-100,
n=0-100,
where n+m is at least equal to 1,
R¹ is a C₁- to C₆-alkyl radical,
R² is a C₁- to C₆-alkyl radical, where R² may be different from R¹,
with the proviso that for at least 50 mol % of the polyalkylene glycol dialkyl ether
m+n is greater than or equal to 11.
R¹ is preferably methyl or ethyl.
R² is preferably methyl or ethyl.
m is preferably from 4 to 60, particularly preferably from 11 to 30.
n is preferably from 1 to 20.
m+n is for at least 50 mol % of the polyalkylene glycol dialkyl ether preferably greater than 12, in particular greater than 13, specifically greater than 14.

The alkylene oxides are preferably ethylene oxide, propylene oxide or butylene oxide, or mixtures thereof.

The amount in which the polyalkylene glycol dialkyl ether can be added is about 0.1-20-fold (% by weight) based on the amount of water employed; preferably 1- to 10-fold (% by weight). The relative molar mass may be between 400 and 12 000 g/mol; preferably 500 to 4000 g/mol.

The polyalkylene glycol dialkyl ethers are known per se or can be prepared by known processes by reacting polyalkylene glycols with an alkylating agent.

A catalyst is not absolutely necessary but can be used to increase the selectivity further. Suitable catalysts are basic compounds such as, for example, alkali metal and alkaline earth metal salts. These catalysts include potassium and sodium hydroxides, potassium and sodium acetates, potassium and sodium phosphates, potassium and sodium halides, potassium and sodium carbonates and the like. The catalyst may be added as salt or be formed in situ.

The process of the invention can be carried out continuously or batchwise. A continuous procedure is preferred. In addition, the process takes place under conditions of temperature and pressure as are usual for industrial processes (Ullmann's Encyclopedia of Industrial Chemistry, Wiley-VCH Verlag, 6th edition, CD-ROM 2003). Temperatures between 80 and 400° C. and a pressure below 50 bar are preferred. A temperature between 100 and 300° C. and a pressure of between 20 and 40 bar are particularly preferred.

The process can be carried out under a CO₂ atmosphere and then presumably proceeds via a carbonate intermediate.

The process of the invention will now be explained in more detail in some examples:

### EXAMPLES

A stainless steel stirred autoclave with a capacity of 500 ccm was used as reactor. The autoclave was equipped with a gas-introduction tube, thermoelectric elements, stirrer, electric heating jacket and cooling coil. During operation, the reactor was charged with a mixture of distilled water, optionally 1% potassium iodide as catalyst, 200 g of polyethylene glycol dimethyl ether having a molecular weight of 540 g/mol (Polyglykol DME 500 from Clariant with an average content of 58% of homologs with n≧11 (determined by gas chromatography)) and either N₂ or CO₂ and heated to the reaction temperature. After the desired reaction temperature was reached, the reactor was charged with ethylene oxide. After a reaction time of 2 hours, the reactor was decompressed and the reaction product was analyzed by gas chromatography.

200 g each of 1,2-dimethoxyethane (monoethylene glycol dimethyl ether from Clariant with n=1), tetraethylene glycol dimethyl ether (from Clariant, n=4) or Polyglykol DME 250 (polyethylene glycol dimethyl ether with a molecular weight of about 240 g/mol and a maximum content of 5% of homologs with n≧11 (determined by gas chromatography)) were used in the comparative examples.

The following table shows the reaction conditions used and the prepared monoethylene glycol (MEG), diethylene glycol (DEG) and triethylene glycol (TEG) in % by weight. The conversion of ethylene oxide to glycols was virtually quantitative.

TABLE 1

| | | | | EO to | | | | | | |
|---|---|---|---|---|---|---|---|---|---|---|
| | Temp. | EO | H₂O | H₂O | Polyalkylene | | | | | |
| No. | [° C.] | [g] | [g] | ratio | glycol diether | Atmosphere | Cat. | MEG | DEG | TEG |
| 1^{a)} | 160 | 20.0 | 72.0 | 1:3.6 | — | N₂ | — | 75.3 | 21.2 | 3.5 |
| 2^{a)} | 160 | 20.0 | 100 | 1:5 | — | N₂ | — | 84.6 | 14.1 | 1.3 |
| 3^{a)} | 160 | 20.0 | 200 | 1:10 | — | N₂ | — | 91.4 | 8.0 | 0.6 |
| 4^{b)} | 160 | 20.0 | 40.0 | 1:2 | Dimethoxyethane | N₂ | Na₂MoO₄ | 87.4 | 11.2 | 1.4 |
| 5^{b)} | 160 | 20.0 | 80.0 | 1:4 | Dimethoxyethane | N₂ | Na₂MoO₄ | 88.6 | 9.3 | 2.1 |
| 6^{b)} | 160 | 20.0 | 46.0 | 1:2.3 | Tetraethylene | N₂ | | 85.3 | 12.8 | 1.9 |
| | | | | | glycol dimethyl | | | | | |
| | | | | | ether | | | | | |
| 7^{b)} | 160 | 20.0 | 46.0 | 1:2.3 | Polyglykol | N₂ | — | 85.3 | 12.8 | 1.9 |
| | | | | | DME 250 | | | | | |
| 8 | 160 | 20.0 | 72.0 | 1:3.6 | Polyglykol | N₂ | — | 97.5 | 2.5 | 0.0 |
| | | | | | DME 500 | | | | | |
| 9 | 160 | 20.0 | 46.0 | 1:2.3 | Polyglykol | N₂ | — | 94.0 | 5.4 | 0.6 |
| | | | | | DME 500 | | | | | |
| 10 | 200 | 20.0 | 72.0 | 1:3.6 | Polyglykol | CO₂ | Kl | 96.2^{c)} | 1.6 | 0.0 |
| | | | | | DME 500 | | | | | |
| 11 | 160 | 20.0 | 46.0 | 1:2.3 | Polyglykol | N₂ | Kl | 96.6 | 2.0 | 1.4 |
| | | | | | DME 500 | | | | | |

^{a)}Comparative examples without Polyglykol DME 500
^{b)}Comparative examples according to US 4760200
^{c)}2.2% ethylene carbonate was detectable in the reaction product.

The examples make it clear that a higher selectivity can be achieved by adding Polyglykol DME 500 than on addition of considerable amounts of water. The energy required to remove the amounts of water from the final product is considerably higher than for removing polyalkylene glycol dimethyl ether. The process of the invention is thus substantially more economical.

1. A process for preparing an alkylene glycol by hydration of alkylene oxide in the presence of polyalkylene glycol dialkyl ether of the formula
R¹—[—(CH₂CH₂O)ₘ(CH(CH₃)CH₂)—O]ₙ—R²
in which
m=0-100,
n=0-100,
where n+m is at least equal to 1,
R¹ is a C₁- to C₆-alkyl radical,
R² is a C₁- to C₆-alkyl radical, where R² may be different from R¹,
with the proviso that for at least 50 mol % of the polyalkylene glycol dialkyl ether
m+n is greater than or equal to 11.

2. The process as claimed in claim 1, in which R¹ is methyl or ethyl.

3. The process as claimed in claim 1, in which R² is methyl or ethyl.

4. The process of claim 1, in which m is 4-60.

5. The process of claim 1, in which n is 1-20.

6. The process of claim 1, in which m+n for at least 50 mol % of the polyalkylene glycol dialkyl ether is greater than 12.

7. The process of claim 1, in which the alkylene oxide is selected from the group consisting of ethylene oxide, propylene oxide or butylene oxide, and mixtures thereof.

8. The process of claim 1, in which the polyalkylene glycol dialkyl ether is from 1 to 20% by weight based on the amount of alkylene oxide employed.
